(19) United States
(12) Patent Application Publication
(10) Pub. No.: US 2006/0178422 A1
(43) Pub. Date: Aug. 10, 2006

(54) Process for making (2S, 3aS, 7aS)-1-[(2S)-2-[[(1S)-1-(ethoxycarbonyl) butyl] amino]-1-oxopropyl] octahydro-1H-indole-2-carboxylic acid

(76) Inventors: Ashok Kumar, (US); Satish Rajanikant Soudagar, (US); Arpana Mathur, (US); Chirag Hasmukh Shah, (US); Sanjay Tukaram Gunjal, Mumbai (IN); Dattatray Shamrao Metil, Thane (IN); Rahul Suresh Kelkar, Mumbai (IN); Devendra Digambar Thakare, Mumbai (IN); Bindu Manoj Kumar, Thane (IN); Raji Nair, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 11/140,226

(22) Filed: May 27, 2005

(30) Foreign Application Priority Data
May 31, 2004 (IN) 566/MUM/2004

(51) Int. Cl. A61K 31/405; C07D 209/42

(52) U.S. Cl. 514/419; 548/492

(57) ABSTRACT

The present invention discloses a process for the synthesis and isolation of (2S, 3aS, 7aS)-1-[(2S)-2-[[(1S)-1-(ethoxycarbonyl)butyl]amino]-1-oxopropyl] octahydro-1H-indole-2-carboxylic acid and its tert-butylamine salt, by condensing (2S, 3aS, 7aS)-octahydroindole-2-carboxylic acid benzyl ester and N[(S)1-carboxybutyl]-(S)-alanine ethyl ester in nonreactive solvents in turn avoiding the formation of impurity viz. N-acetyl (2S,3aS,7aS)-octahydroindole-2-carboxylic acid benzyl ester (Formula V). The de-protection of benzyl ester group is optimized and then isolation of the product from aqueous layer by extraction using an organic solvent, which eliminates the need of lyophilization. The process of the present invention yields perindopril erbumnine salt of Formula 1B free of contaminants derivable from dicyclohexylcarbodiimide and impurities originated by the use of ethyl acetate.

### FIELD OF THE INVENTION

The present invention relates to a practical, plant friendly and economical process for the manufacture of (2S, 3aS, 7aS)-1-[(2S)-2-[[(1S)-1-(ethoxycarbonyl)butyl]amino]-1-oxopropyl]octahydro-1H-indole-2-carboxylic acid herein after called Perindopril or more particularly to the process for the manufacture of its pharmaceutically acceptable salt viz. tert. butyl amine salt herein after called perindopril erbumine having high purity.

### BACKGROUD

Perindopril (Formula IA) and its pharmaceutically acceptable salts, especially the tert. butylamine salt (Formula IB), have valuable pharmacological properties. Their main property lies in the inhibition of the enzyme that converts angiotensin I (or kininase II), a precursor for formation of angiotensin II enzyme, thereby enables on the one hand prevention of the conversion of the decapeptide angiotensin I to the octapeptide angiotensin II (vasoconstrictor) and on the other hand prevention of the degradation of bradykinin (vasodilator) to inactive peptide. These two actions contribute to the beneficial effects of perindopril or its salts in cardiovascular disorders, especially arterial hypertension and cardiac insufficiency. The use of perindopril in these therapies demands high purity of the final compound in a manufacturing operation.

Perindopril and its therapeutic use were first described in European Patent Specification No. 0049658.

An alternative route of synthesis for perindopril has been reported in Tetrahedron Letters 23, (16), 1677-1680, (1982), wherein the tert. butyl ester of (2S, 3aS, 7aS)-octahydroindole-2-carboxylic acid was coupled with N-[(S)-1-carboxybutyl]-(S)-alanine ethyl ester (Formula III) in presence of triethyl amine, dicyclohexylcarbodiimide, and 1-hydroxy benzotriazole and subsequently deprotecting the tert. butyl ester group. Tert. butyl amine salt of perindopril was reported for the first time in the above publication (Page No1679. lines 11-12).

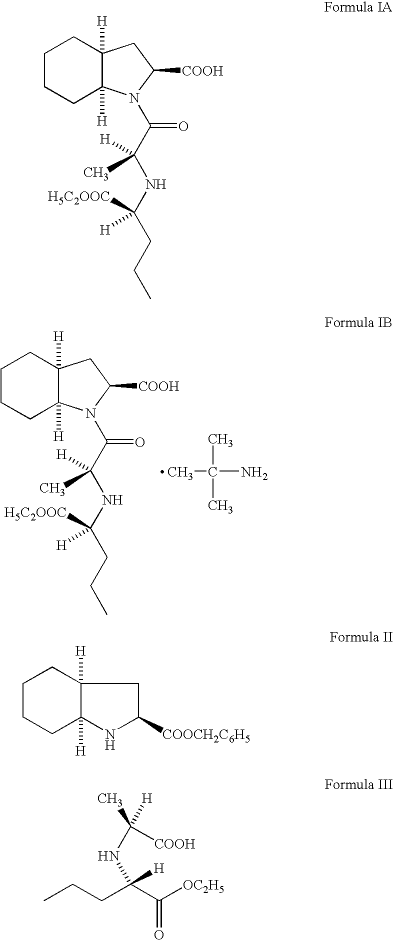

The first industrial synthesis of perindopril was disclosed in the European patent No. 0308341 by the reaction of (2S, 3aS, 7aS)-octahydroindole-2-carboxylic acid esters of Formula II with N-[(S)-1-carboxybutyl]-(S)-alanine ethyl ester of Formula III using triethylamine, dicyclohexylcarbodiimide and 1-hydroxy benzotriazole to give the compound of Formula IV followed by de-protection of ester group selectively by methods known in the art to get perindopril (Formula IA). Perindopril erbumine salt (Formula IB) is then obtained by combining tert. butylamine with Perindopril (free acid).

However, the product obtained by the above process, contains many impurities, as also observed by others and the process improvement taking care of a part of the problem has been subject of patent applications No. US 2003/0069431 and WO 0364388.

The U.S. patent application 2003/0069431 describes the modified process for the manufacture of Perindopril and its tert.-butyl amine salt. It discloses reaction of benzyl ester of (2S, 3aS, 7aS)-octahydroindole-2-carboxylic acid with N-[(S)-1-carboxybutyl]-(S)-alanine ethyl ester in ethyl acetate, in presence of reduced molar 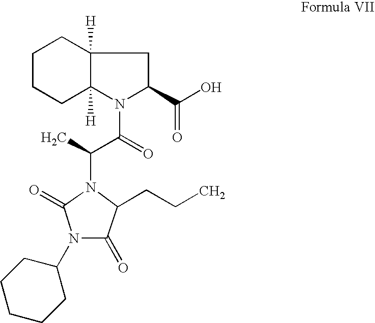
quantities of 1-hydroxy benzotriazole, dicyclohexyl carbodoiimide and in presence or absence of triethyl amine, whereby the impurities of Formula VII & Formula VIII in perindopril are brought down to below 0.1 and 0.2% respectively.

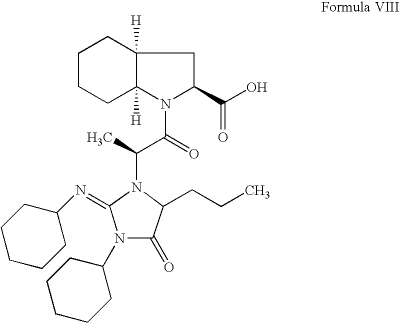

According to the process mentioned in patent application No. WO 03/064388, the compound of Formula III is Oveprotected by suitable carbonic acid derivatives. The carboxylic acid group is then converted into its acid chloride, followed by its reaction with octahydroindole-2-carboxylic acid (Formula H), yielding perindopril of better purity. It also avoids use of dicyclohexyl carboduimide. This patent also addresses the problems in the prior art. Various other process-patents such as EP1371659, EP1380591, EP1380590, EP1362864, EP1367061 are published, and claim to minimize the problems associated with prior art i.e. purity of Perindopril. In these patents, the route of synthesis and the intermediates are different than the patents, discussed above.

Thorough study of prior art indicates that the process disclosed in EP0308341 (U.S. Pat. No. 4,914,214) is best suited as an industrial route and comparatively more economical to produce Perindopril, provided it avoids the formation of impurities as well as eliminates need for additional purification step. Patent application WO 01/58868 addresses this problem to an extent by reducing two impurities having Formula VII and VIII to give improved quality of the product.

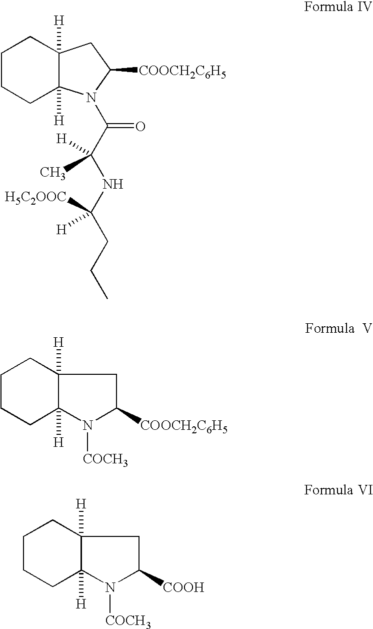

The processes mentioned in patents EP 0308341 and WO 01/58868 describe the use of ethyl acetate as a solvent in the coupling step. Under the conditions of coupling, one major impurity viz. N-acetyl (2S, 3aS, 7aS)-octahydroindole-2-carboxylic acid benzyl ester (Formula V) is formed. Formation of this impurity has been identified to be associated with the use of ethyl acetate as solvent. The reason being ethyl acetate acting as acylating agent to form the impurity of Formula V. Removal of this impurity is very difficult at this stage, as the physical properties of the impurity and the coupled products are very similar in nature. It is also difficult to remove the impurity in the next step i.e. debenzylation. The coupled product (Formula IV) gives Perindopril (Formula IA) whereas compound of Formula V also gets debenzylated to form N-acetyl (2S, 3aS, 7aS) octahydroindole-2-carboxylic acid (Formula VI), which is an impurity. The heterocyclic part in Perindopril(Formula IA) and this impurity (Formula VI), is also same, i.e. octahydrocarboxy indole. Due to this, during salt formation of Perindopril with tert.-butylamine, compound of Formula VI invariably remains contaminated with the product (Formula IB).

Further problem is associated with the incomplete removal of the impurity of dicyclohexyl urea. Use of dicyclohexyl carbodiimide leads to formation of dicyclohexyl urea as byproduct, and needs to be filtered off. However, the traces of it always get carried over in subsequent steps.

Another major drawback of the prior art (EP 0308341 and WO 01/58868) is the debenzylation of Formula IV by hydrogenation using catalysts such as palladium on carbon in the two phase media like water and cyclohexane or methylcyclohexane and water. Use of two-phase solvents during hydrogenation leads to reduced rate of reaction and longer time in the completion of the reaction. Separation of catalyst by filtration also becomes slow due to biphasic system.

Yet another major drawback of the prior art lies in the isolation technique of perindopril from aqueous media. In all of them, isolation of product from aqueous solution is carried out by lyophilization. Though lyophilizers are used by industry, they are not preferred techniques, as far as large-scale operations are concerned. It also means heavy investments in capital assets.

From the teaching of the prior art and our own studies, it is amply clear that there remains ample scope to further develop process for Perindopril, which is of high purity and eliminate the need for lyophilization. The lacuna and scope in the prior art lead us to carry out the present invention.

### OBJECTS OF THE INVENTION

The main objective of the present invention is to develop a manufacturing process for perindopril and its salts like tertiary butylamine salt having high purity, low impurities and involving simple isolation technique. Other objectives of the present invention are 1) to provide a process for the preparation of high purity perindopril free of impurities generated in the coupling reaction viz. impurity of Formula V, 2) to provide a process wherein the contamination of the product, derived from dicyclohexyl carbodiimide particularly dicyclohexyl urea is minimized, 3) to develop simple process for isolation of perindopril using solvent extraction methods and in turn eliminating the need for lyophilization.

Other objectives and advantages will become apparent to persons skilled in the art and familiar with the background references from a careful reading of this specification.

### SUMMARY OF THE INVENTION

The present invention discloses a process for the synthesis and isolation of (2S, 3aS, 7aS)-1-[(2S)-2-[[(1S)-1-(ethoxycarbonyl)butyl]amino]-1-oxopropyl]octahydro-1H-indole-2-carboxylic acid and its tert. butylamine salt, by condensing (2S, 3aS, 7aS)-octahydroindole-2-carboxylic acid benzyl ester and N[(S)-1-carboxybutyl]-(S)-alanine ethyl ester in nonreactive solvents other than ethyl acetate, such as N,N-dimethyl formamide, acetonitrile, methylene chloride, chloroform, cyclohexane, in turn avoiding the formation of impurity viz. Nacetyl (2S,3aS,7aS)-octahydroindole-2-carboxylic acid benzyl ester (Formula V) followed by de-protection of benzyl ester group by hydrogenation using catalyst like palladium on charcoal in alcoholic solvents such as ethanol or isopropyl alcohol and then isolation of the product from aqueous layer by extraction using an organic solvent, which eliminates the need of lyophilization.

According to a particular embodiment of present invention there is provided compound of Formula IV, free of contamination of dicyclohexyl urea by dissolving compound of Formula IV in diisopropyl ether or cyclohexane or a mixture thereof and then filtering off dicyclohexyl urea.

The solvent used in the deprotection/hydrogenation is alcoholic solvent preferably ethanol and/or isopropanol. The compound (2S,3aS,7aS)-1-[(2S)-2-[[(1S)-1-(ethoxycarbonyl)butyl]amino]-1-xopropyl]octahydro-H-indole-2-carboxylic acid obtained after hydrogenation is isolated by extraction from aqueous medium by using the solvents such as ethyl acetate or methylene chloride.

The compound (2S, 3aS, 7aS)-1-[(2S)-2-[[(1S)-1-(ethoxycarbonyl)butyl]amino]-1-xopropyl]octahydro-H-indole-2-carboxylic acid is isolated in solid form by making slurry in non-polar organic solvent such as hexane or cyclohexane or mixtures thereof.

### DETAILED DESCRIPTION OF THE INVENTION

The present invention relates to a process for the synthesis of (2S, 3aS, 7aS)-1-[(2S)-2-[[(1S)-1-(ethoxycarbonyl)butyl]amino]-1-oxopropyl]octahydro-1H-indole-2-carboxylic acid (Formula IA) by the reaction of compound of Formula II with the compound of Formula III in presence of reagents such as dicyclohexyl carbodiimide, 1-hydroxybenzotriazole & triethyl amine at a temperature from 20-30° C. in a solvent selected from N,N-dimethyl formamide, acetonitrile, methylene chloride, chloroform and cyclohexane.

It has been observed that use of solvent like ethyl acetate, leads to the formation of impurity of Formula V. Ethyl acetate acts as acylating agent under the above reaction conditions. Therefore, use of ethyl acetate as a solvent is avoided. By following the present invention, formation of this impurity is completely avoided. This is achieved by selecting solvents such as N,N-dimethyl formamide, acetonitrile, methylene chloride, chloroform, and cyclohexane, which can not act as acylating agent. The most preferred solvent used is methylene chloride.

The molar ratios used for compound of Formula II & III were in the range of 1:1.0 to 1:1.2, where the most preferred ratio is 1:1.2. The dicyclohexyl carbodiimide is used in molar ratio of 1:1.0 to 1:1.2 with reference to compound of Formula II, where the most preferred ratio is 1:1.2. 1-Hydroxybenzotriazole is used in molar ratio of 1:1.0 to 1:1.2 with reference to compound of Formula II, where the most preferred ratio used is 1:1.1. Triethyl amine is used in molar ratio of 1:1 to 1:3 with reference to compound of formula H, wherein the most preferred ratio used is 1:3 moles.

The byproduct, dicylohexyl urea, of dicyclohexylcarbodiimide formed during the reaction is conventionally filtered during work up. However, the removal of last traces of dicyclohexyl urea is difficult and always traces remains with the product. The present invention provides a simple solution to this problem, which includes filtration of insoluble material after the completion of the reaction followed by dilution of the mother liquor with water, followed by separation of organic and aqueous layers. From the organic layer solvent is distilled out under reduced pressure. The residue obtained is dissolved in solvent like diisopropyl ether or cyclohexane wherein the most preferred solvent is diisopropyl ether. The trace impurity of dicyclohexyl urea remained insoluble, is filtered off. The product of Formula IV obtained after removal of solvent is free from the impurities of Formula V and dicyclohexyl urea.

The product of Formula IV thus obtained is then subjected to deprotection of benzyl group by hydrogenation in presence of catalyst such as palladium on charcoal using alcoholic solvent like ethanol or isopropyl alcohol. The hydrogenation is carried out in the temperature range of 25° C. to 40° C. wherein the preferred temperature is 30° C.

Catalyst used is 5% to 10% palladium on charcoal, wherein preferred catalyst is 5% palladium on charcoal. Hydrogen pressure used during reduction is atmospheric pressure to 5 kg/cm2 wherein the preferred pressure is 2 kg/cm2.

At the end of reaction, the catalyst is filtered off. The advantage of the present invention is that not only the rate of debenzylation is faster than biphasic system but the rate of filtration of the catalyst is also much efficient.

Isolation of Perindopril & Converting it to Erbumine Salt:

From the above filtrate containing perindopril, the alcoholic solvent is distilled out under reduced pressure. The residue obtained is treated with the mixture of cyclohexane: water in the ratio preferably 50:50. Organic layer is separated and the aqueous layer containing perindopril is extracted by solvents like ethyl acetate or methylene chloride. Upon distillation of the solvent the product obtained is of the Formula IA, which is a semisolid viscous mass. This gets solidified by simply triturating with nonpolar solvent like cyclohexane or hexane or mixture thereof.

The isolation and purification of compound of Formula IA according to the present invention is particularly important because the product is directly extracted in solvent, which is very simple to operate. Apart from the operation-wise advantages and industrial scalability of the solvent extraction process; the perindopril obtained with these operations is pure because almost all polar impurities remain in aqueous medium and thus do not contaminate the precious product.

Perindopril erbumine is prepared from its free acid by the process given in the prior art (EP 0308341), i.e. by combining perindopril acid obtained in the above mentioned isolation process with tert. butyl amine in ethyl acetate and the mixture is heated to get clear solution. The mixture is cooled to a temperature of 25-30° C. to allow the crystallization of the perindopril erbumine salt, similar to the reported α-form as characterized by XRD study.

Given below are the examples to illustrate the invention but do not limit the scope of invention in any way.

### EXAMPLES

### Example—I

### (2S, 3aS, 7aS)-1-{(2S)-2-(1S)-1-(Ethoxycarbonyl)-butylamino]-propionyl}-1H-indole-2-benzyl carboxylate

To a suspension of 100 gm. of para-toluene sulfonate of benzyl ester of (2S, 3aS, 7aS)-octahydroindole-2-carboxylic acid in 2.0 litre of methylene chloride, 70.3 gm of triethylamine is added at 20-25° C. After stirring, 34.5 gm of 1-hydroxybenzotriazole, 60.4 gm. of N-[(S)-carbethoxy-1-butyl]1(S)-alanine and 57.4 gm. of dicyclohexylcarbodiimide were added in the same sequence at the interval of about 15 minutes.

The heterogeneous mass is stirred till completion of reaction at 20-25° C. Then the dicyclohexyl urea is filtered and the filtrate is washed with water. The solvent is removed under vacuum. Approx. 1.0 litre of diisopropyl ether is added to the above mass and stirred for about 15 minutes, filtered, solvent distilled under vacuum to give 105 gm. (99%) product in the form of oil.

### Example—II

### (2S, 3aS, 7aS)-1-[(2S)-2-[[(1S)-1-(ethoxycarbonyl)butyl]amino]-1-oxopropyl]octahydro-1H-indole-2-carboxylic acid (perindopril acid)

The oily material (100 gm) obtained in the preceding stage is dissolved in about 1.0 litre absolute ethanol in a hydrogenator. 10 gm Palladium on charcoal catalyst (5%) is added to the above solution. The mixture is hydrogenated under pressure of about 2 kg./cm2 at ambient temperature till completion of reaction. The catalyst is filtered and the solvent is removed under vacuum to get oily mass. This is suspended in about 500 ml. of water. The aqueous layer is thoroughly washed with cyclohexane. The product is extracted in ethyl acetate from the aqueous phase. Ethyl acetate is distilled and the mass is stirred with cyclohexane and then filtered to get 50 gm (63%) of solid.

### Example—III

### Tert. Butylamine salt of (2S, 3aS, 7aS)-1-[(2S)-2-[[(1S)-1-(ethoxycarbonyl)butyl]amino]-1-oxopropyl]octahydro-1H-indole-2-carboxylic acid

50 gm perindopril free acid obtained as above is converted to salt by adding 11.0 gm. of tert. butylamine in 750 ml ethyl acetate and the mixture is heated till clear solution obtained. It is cooled to 25-30° C. and filtered to get perindopril erbumine 54 gm (90%).

### Example—IV

Oily mass (about 100 gm) obtained as per the Example-II above, is suspended in about 500 ml of water. The aqueous layer is thoroughly washed with cyclohexane. The product is extracted in ethyl acetate and concentrated to 750 ml. To it tert. Butyl amine (11.0 gm) is added. The mixture is heated till clear solution obtained. It is then cooled and filtered to get perindopril erbumine 54 gm (90%).

Given our disclosure, one of skill in the art can readily modify our invention. For example, the process may be used to make an α-form of the product compound. We therefore intend our patent to have legal coverage as defined by the appended claims, rather than be limited to any specific example discussed above.

We claim:

1. A manufacturing process for perindopril erbumine salt of Formula IB comprising: 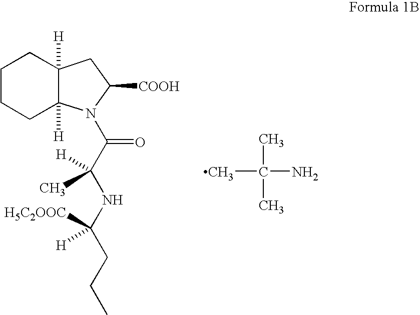
i. reacting a compound of Formula II with a compound of Formula III (1.0-1.2 moles) in presence of (1.0-1.2 moles) of dicyclohexylcarbodiimide, (1.0-1.2 moles) of 1-hydroxybenzotriazole and 1.0-3.0 moles of triethyl amine characterized in that the coupling reaction is carried out in a non-reactive solvent other than ethyl acetate and isolating (2S,3aS,7aS)-1-{(2S)-2-(1S)-1-(Ethoxycarbonyl)-butylamino]-propionyl}-octahydro-1H-indole-2-benzyl carboxylate free of contamination due to impurity of Formula V and dicyclohexyl urea;
ii. deprotecting (2S, 3aS, 7aS)-1-{(2S-2-(1S)-1-(Ethoxycarbonyl)-butylamino]-propionyl}-octahydro-1H-indole-2-benzyl carboxylate using palladium on charcoal as catalyst in a single solvent system and isolating by extraction in organic solvent from aqueous medium followed by distilling solvent to get (2S, 3aS, 7aS)-1-[(2S)-2-[[(1S)-1-(ethoxycarbonyl)butyl]amino]-1-oxopropyl]octahydro-1H-indole-2-carboxylic acid in solid form;
iii. combining (2S, 3aS, 7aS)-1-[(2S)-2-[[(1S)-1-(ethoxycarbonyl)butyl]amino]-1-oxopropyl]octahydro-1H-indole-2-carboxylic acid with equimolar quantity of tertiary butyl amine in a solvent to yield high purity perindopril erbumine.

2. A process for manufacture of perindopril erbumine as claimed in claim 1 wherein, the said nonreactive solvents used in the coupling reaction are selected from methylene chloride, chloroform, acetonitrile, N,N-dimethyl formamide and cyclohexane.

3. A process for manufacture of perindopril erbumine as claimed in claim 1 and 2 wherein, the most preferable non-reactive solvent used in the coupling reaction is methylene chloride.

4. A process for manufacture of perindopril erbumine as claimed in claim 1 wherein, the removal of dicyclohexyl urea is effected in diisopropyl ether, cyclohexane or a mixture thereof.

5. A process for manufacture of perindopril erbumine as claimed in claim 1 wherein, the said solvent used in the deprotection/hydrogenation is alcoholic solvent.

6. A process for manufacture of perindopril erbumine as claimed in claim 1 and 5 wherein, the said alcoholic solvent used in the deprotection/hydrogenation is ethanol.

7. A process for manufacture of perindopril erbumine as claimed in claim 1 and 5 wherein, the alcoholic solvent used in the deprotection/hydrogenation is isopropyl alcohol.

8. A process for manufacture of perindopril erbumine as claimed in claim 1 wherein, the compound (2S,3aS,7aS)-1-[(2S)-2-[[(1S)-1-(ethoxycarbonyl)butyl]amino]-1-xopropyl]octahydro-1H-indole-2-carboxylic acid obtained after hydrogenation is isolated by extraction from aqueous medium.

9. A process for manufacture of perindopril erbumine as claimed in claim 8 wherein, the extraction is carried out using solvents like ethyl acetate or methylene chloride.

10. A process for manufacture of perindopril erbumine of claim 9 wherein the compound (2S, 3aS, 7aS)-1-[(2S)-2-[[(1S)-1-(ethoxycarbonyl)butyl]amino]-1-xopropyl]octahydro-1H-indole-2-carboxylic acid is isolated in solid form by making slurry in nonpolar organic solvent.

11. A process for manufacture of perindopril erbumine of claim 9 and 10 wherein the compound (2S,3aS,7aS)-1-[(2S)-2-[[(1S)-1-(ethoxycarbonyl)butyl]amino]-1-xopropyl]octahydro-H-indole-2-carboxylic acid isolated in solid form by making slurry in hexane or cyclohexane or mixture thereof.

12. A process for manufacture of perindopril erbumine as substantially described herein with reference to the foregoing examples 1 to 4.
